SEQUENCE LISTING (2 sequences)

SEQ ID NO: 1 - DNA, 13 bp, Artificial Sequence
  chemically synthesized oligonucleotide
tcgnntcgnntcg

SEQ ID NO: 2 - DNA, 24 bp, Artificial Sequence
  chemically synthesized oligonucleotide
tcgtcgttttgtcgttttgtcgtt

(19) United States
(12) Patent Application Publication
(10) Pub. No.: US 2008/0008741 A1
(43) Pub. Date: Jan. 10, 2008

(54) Methods of Treating Irritable Bowel Syndrome

(76) Inventors: Eyal Raz, Del Mar, CA (US); Daniel Rachmilewitz, Jerusalem (IL)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/588,573

(22) PCT Filed: Jan. 27, 2005
(86) PCT No.: PCT/US05/02594

§ 371 Date: May 1, 2007

Related U.S. Application Data
(60) Provisional application No. 60/541,861, filed on Feb. 3, 2004.

(51) Int. Cl. A61K 31/7088; A61K 47/00

(52) U.S. Cl. 424/439; 514/44

(57) ABSTRACT

The present invention provides methods of treating irritable bowel syndrome (IBS) in an individual suffering from IBS. The methods generally involve administering to the individual an effective amount of a therapeutic nucleic acid. The invention further provides kits and compositions for practicing the subject methods.

### CROSS-REFERENCE

This application claims the benefit of U.S. Provisional Patent Application No. 60/541,861 filed Feb. 3, 2004, which application is incorporated herein by reference in its entirety.

### STATEMENT REGARDING FEDERALLY SPONSORED RESEARCH

The U.S. government may have certain rights in this invention, pursuant to grant no. AI40682 awarded by the National Institutes of Health.

### FIELD OF THE INVENTION

The present invention is in the field of treatments for irritable bowel syndrome.

### BACKGROUND OF THE INVENTION

Irritable bowel syndrome (IBS) is a common disorder, which affects up to 20% of the population worldwide. Some studies suggest that only about 10% to 50% of those afflicted with IBS actually seek medical attention. Nonetheless, IBS still accounts for up to about 3.5 million physician visits per year, and is the most common diagnosis in a gastroenterologists' practice, accounting for about 25% of all patients. IBS is a functional bowel disorder in which abdominal pain is associated with defecation or a change in bowel habits. A hallmark of IBS is abdominal pain that is relieved by defecation, and which is associated with a change in the consistency or frequency of stools. IBS may be diarrhea-predominant, constipation-predominant, or an alternating combination of both. There is currently no mechanical, biochemical, or overt inflammatory condition that explains the symptoms.

The medical community has developed a consensus definition and criteria, known as the Rome criteria, to aid in diagnosis of IBS. According to the Rome criteria, IBS is indicated by abdominal pain or discomfort which is (1) relieved by defection and/or (2) associated with a change in frequency or consistency of stools, plus two or more of the following: altered stool frequency, altered stool formation, altered stool passage, passage of mucus, and bloating or feeling of abdominal distention (Dalton and Drossman (1997) Am. Fam. Physician 55(3):875-880).

Current treatments for IBS include pharmacological and non-pharmacological therapies. Pharmacological strategies include antispasmodic agents, tricyclic antidepressants, serotonin 5HT3 receptor antagonists, serotonin 5HT4 receptor agonists, and other agents such as antibodies, herbs, peppermint oil, and probiotics. Non-pharmacological therapies currently in use for treatment of IBS include elimination diets, exercise, supportive psychosocial advice and lifestyle modification.

There is an ongoing need for new methods for treating IBS. The present invention addresses this need.

### Literature

U.S. Pat. Nos. 6,638,928; 6,284,770; 6,228,040; 6,203,797; 6,613,751; 6,194,382; 5,965,557; 5,900,233; 5,840,332; 6,127,418; and 6,297,226; Kim et al. (2003) Aliment. Pharmacol. Ther. 17:895; Mertz et al. (2003) New Engl. J. Med. 349:2136-2146; U.S. Patent Publication No. 20030119756; Hicks et al. (2002) J. Physiol. 544:861-869; Kobayashi et al. (2001) Jpn. J. Pharmacol. 86:281-288; Al-Chaer et al. (2000) Gastroenterol. 119:1276-1285; Barbara et al. (2002) Gut 51(Suppl I):i41-i44; U.S. Patent Publication No. 20040013741.

### SUMMARY OF THE INVENTION

The present invention provides methods of treating irritable bowel syndrome (IBS) in an individual suffering from IBS. The methods generally involve administering to the individual an effective amount of a therapeutic nucleic acid. The invention further provides kits and compositions for practicing the subject methods.

### DEFINITIONS

The term “ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation. By “treatment” is meant at least a reduction or an amelioration of at least one of the symptoms associated with the disease condition afflicting the host, where amelioration is used in a broad sense to refer to at least a reduction in the magnitude of a parameter, e.g. symptom, associated with the pathological condition being treated, such as bloating and pain associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition. “Treatment” or “treating” as used herein means any therapeutic intervention in a mammalian subject, e.g., a human subject, including: (i) prevention, that is, causing the clinical symptoms not to develop, e.g., preventing progression to a harmful state; (ii) inhibition, that is, arresting the development or further development of clinical symptoms; and/or (iii) relief, that is, causing the regression of clinical symptoms, e.g., causing relief from diarrhea, constipation, abdominal pain, etc.

As used herein, “subject” or “individual” or “patient” refers to any subject for whom or which therapy is desired, and generally refers to the recipient of the therapy to be practiced according to the invention. The subject can be any vertebrate, but will generally be a mammal, often a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a therapeutic nucleic acid” includes a plurality of such nucleic acids and reference to “the treatment regimen” includes reference to one or more treatment regimens and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating irritable bowel syndrome (IBS) in an individual suffering from IBS. The methods generally involve administering to the individual an effective amount of a therapeutic nucleic acid. In some embodiments, the methods further involve administering at least a second therapeutic agent.

In some embodiments, an effective amount of a therapeutic nucleic acid is an amount that is effective to reduce at least one symptom of IBS by at least about 10%, at least about 20%, at least about 30%, at least about 40%, at least about 50%, at least about 60%, at least about 70%, at least about 80%, or at least about 90%, or more, compared to the symptom in the absence of treatment with the therapeutic nucleic acid. Thus, for example, an effective amount of a therapeutic nucleic acid reduces abdominal pain by at least about 10%, at least about 20%, at least about 30%, at least about 40%, at least about 50%, at least about 60%, at least about 70%, at least about 80%, or at least about 90%, or more, compared to the abdominal pain in the absence of treatment with the therapeutic nucleic acid. In some embodiments, where the patient is experiencing constipation, an effective amount of a therapeutic nucleic acid reduces constipation by at least about 10%, at least about 20%, at least about 30%, at least about 40%, at least about 50%, at least about 60%, at least about 70%, at least about 80%, or at least about 90%, or more, compared to the degree of constipation in the absence of treatment with the therapeutic nucleic acid. In other embodiments, where the patient is experiencing diarrhea, an effective amount of a therapeutic nucleic acid reduces diarrhea by at least about 10%, at least about 20%, at least about 30%, at least about 40%, at least about 50%, at least about 60%, at least about 70%, at least about 80%, or at least about 90%, or more, compared to the degree of diarrhea in the absence of treatment with the therapeutic nucleic acid.

Whether a given therapeutic nucleic acid is effective in treating IBS is readily determined by those skilled in the art. For example, according to the Rome criteria, IBS is indicated by abdominal pain or discomfort which occurs over a period of 12 weeks or more, and which is (1) relieved by defection and/or (2) associated with a change in frequency and/or consistency of stools, plus two or more of the following: altered stool frequency, altered stool formation, altered stool passage, passage of mucus, and bloating or feeling of abdominal distention (Dalton, C. and Drossman, D. A., Am. Fam. Physician 1997 55(3):875-880; and Drossman et al. (1997) Gastroenterology 112:2120-2137).

Diagnostic tests that may be performed are generally conducted to exclude the possibility that the symptoms are caused by a disorder other than IBS. For example, a complete blood count is conducted to check for anemia, inflammation, infection; erythrocyte sedimentation rate screens for inflammation and possible malignancy; hemoccult assay to screen for gastrointestinal bleeding; examination of stool samples for intestinal parasites, enteric pathogens, leukocytes, occult blood, etc.; sigmoidoscopy or colonoscopy to determine inflammation or distal obstruction; barium enema to screen for tumors, inflammation, obstruction, Crohn disease; lactose intolerance tests; and the like.

A therapeutic nucleic acid can be tested in an animal model of IBS for efficacy in reducing one or more symptoms of IBS. Animal models of IBS have been described in the literature. See, e.g., Al-Chaer et al. (2000) Gastroenterology 119:1276-1285; and Kobayashi et al. (2001) Jpn. J. Pharmacol. 86:281-288; Williams et al. (1988) Gastroenterology 94:611-621); and U.S. Pat. No. 6,638,928.

A therapeutic nucleic acid can be administered to a subject prior to onset of symptoms (e.g., prior to onset of abdominal pain), or after onset of symptoms (e.g., after onset of abdominal pain, after onset of constipation, after onset of diarrhea). As such, a therapeutic nucleic acid can be administered at any time, and may be administered at any interval. In one embodiment, a therapeutic nucleic acid is administered about 8 hours, about 12 hours, about 24 hours, about 2 days, about 4 days, about 8 days, about 16 days, about 30 days or 1 month, about 2 months, about 4 months, about 8 months, or about 1 year after initial onset of IBS-associated symptoms in the subject.

In some embodiments, a single dose of a therapeutic nucleic acid is administered. In other embodiments, multiple doses of a therapeutic nucleic acid are administered. For example, in some embodiments, a therapeutic nucleic acid is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid).

A therapeutic nucleic acid is administered for a desired treatment duration, which may vary, depending at least in part on the response of the patient to the agent, the severity of the symptoms, etc. For example, in some embodiments, a therapeutic nucleic acid is administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, a subject method for treating IBS in an individual comprises administering to the individual an effective amount of a therapeutic nucleic acid, where the therapeutic nucleic acid comprises a nucleotide sequence of the formula 5′ CG 3′, and where the therapeutic nucleic acid is delivered orally.

In some embodiments, a subject method for treating IBS in an individual comprises administering to the individual an effective amount of a therapeutic nucleic acid, where the therapeutic nucleic acid comprises a nucleotide sequence of the formula 5′ (TCG)n 3′, where n is any integer that is 1 or greater, and where the therapeutic nucleic acid is delivered orally.

In some embodiments, a subject method for treating IBS in an individual comprises administering to the individual an effective amount of a therapeutic nucleic acid, where the therapeutic nucleic acid comprises a nucleotide sequence of the formula 5′ CG 3′, and where the therapeutic nucleic acid is delivered subcutaneously.

In some embodiments, a subject method for treating IBS in an individual comprises administering to the individual an effective amount of a therapeutic nucleic acid, where the therapeutic nucleic acid comprises a nucleotide sequence of the formula 5′ (TCG)n 3′, where n is any integer that is 1 or greater, and where the therapeutic nucleic acid is delivered subcutaneously.

### Combination Therapy

In some embodiments, a subject method is modified to include administration of one or more additional therapeutic agents. Suitable additional therapeutic agents include, but are not limited to, serotonin 5HT3 receptor antagonists; serotonin 5HT4 receptor agonists; somatostatin analogs; muscarinic receptor antagonists; laxatives; antispasmodics; antidepressants; antidiarrheal agents; prokinetic agents; peripheral opiate narcotic antagonists; and the like. Suitable 5-HT3 serotonin receptor antagonists include, but are not limited to, Alosetron (Lotronox), renzapride cilansetron, ondansetron, and the like. Suitable 5-HT4 agonists include, but are not limited to, Tegaserod (Zelnorm), Prucalopride, renzapride, and the like. Suitable somatostatin analogs include, but are not limited to, Octreotide. Suitable muscarinic receptor antagonists include, but are not limited to, Darifenacin, Zamifenacin, and the like. Suitable laxatives include, but are not limited to, methylcellulose (Citrucel), Psyllium (Metamucil, Fiberall, Reguloid, Konsyl), malt soup extract, polyacrylic resins (e.g., hydrophilic forms such as polycarbophil and calcium polycarbophil), plantago seeds, dioctyl calcium sulfosuccinate, dioctyl potassium sulfosuccinate, dioctyl sodium sulfosuccinate, mineral oil, magnesium citrate, magnesium hydroxide, magnesium sulfate, dibasic sodium phosphate, monobasic sodium phosphate, sodium biphosphate, glycerin, anthraquinones or anthracene laxatives (such as aloe, cascara sagrada, danthron, senna, aloin, casanthranol, frangula, and rhubarb), diphenylmethanes (such as bisacodyl and phenolphthalein), and castor oil and the like. Suitable antispasmodic agents include, but are not limited to, anticholinergic agents such as dicyclomine HCl (Bentyl), hyoscyamine sufate (Levsin), and the like. Suitable antidepressants include, but are not limited to, tricyclic antidepressants such as Imipramine (Tofranil), amitriptylin (Elavil). Suitable antidiarrheal agents include, but are not limited to, diphenoxylate HCl+atropine sulfate (Lomotil), loperamide (Imodium), natural or synthetic opiates (such as difenoxin, diphenoxylate, pargoric, opium tincture, and loperamide), anticholinergics (such as belladonna alkoloids-atropine hyoscyamine, and hyosine), acetyltannic acid, albumin tannate, alkofanone, aluminum salicylates, catechin, lidamidine, mebiquine, trillium, and uzarin, and the like. Suitable peripheral opiate narcotic antagonists include, but are not limited to, Fedotozine, Trimebutine, and the like. Suitable prokinetic agents include, but are not limited to, Cisapride monohydrate (Propulsid), metoclopromide, domperidone, and the like.

In particular embodiments, e.g., where an individual having IBS experiences diarrhea, a subject method comprises administering a dosage of a therapeutic nucleic acid containing an amount of from about 1 μg to about 500 mg therapeutic nucleic acid; and a dosage of dicyclomine HCl containing an amount from about 10 mg to about 40 mg orally qid, for the desired treatment duration.

In particular embodiments, e.g., where an individual having IBS experiences diarrhea, a subject method comprises administering a dosage of a therapeutic nucleic acid containing an amount of from about 1 μg to about 500 mg therapeutic nucleic acid; and a dosage of hyoscyamine sulfate containing an amount from about 0.125 to about 0.25 mg orally every 4 hours, for the desired treatment duration.

In particular embodiments, e.g., where an individual having IBS experiences diarrhea, a subject method comprises administering a dosage of a therapeutic nucleic acid containing an amount of from about 1 μg to about 500 mg therapeutic nucleic acid; and a dosage of Lomotil (diphenoxylate HCl+atropine sulfate) orally qid, for the desired treatment duration.

In particular embodiments, e.g., where an individual having IBS experiences diarrhea, a subject method comprises administering a dosage of a therapeutic nucleic acid containing an amount of from about 1 μg to about 500 mg therapeutic nucleic acid; and a dosage of loperamide containing an amount of 4 mg orally after first loose stool, the 2 mg after each subsequent stool, for the desired treatment duration.

In particular embodiments, e.g., where an individual having IBS experiences constipation, a subject method comprises administering a dosage of a therapeutic nucleic acid containing an amount of from about 1 μg to about 500 mg therapeutic nucleic acid; and a dosage of Tegaserod containing an amount of 6 mg orally bid, for the desired treatment duration.

### Therapeutic Nucleic Acids

The terms “polynucleotide,” and “nucleic acid,” as used interchangeably herein in the context of therapeutic nucleic acid molecules, is a polynucleotide as defined above, and encompasses, inter alia, single- and double-stranded oligonucleotides (including deoxyribonucleotides, ribonucleotides, or both), modified oligonucleotides, and oligonucleosides, alone or as part of a larger nucleic acid construct, or as part of a conjugate with a non-nucleic acid molecule such as a polypeptide. Thus a therapeutic nucleic acid may be, for example, single-stranded DNA (ssDNA), double-stranded DNA (dsDNA), single-stranded RNA (ssRNA) or double-stranded RNA (dsRNA). Therapeutic nucleic acids also encompasses crude, detoxified bacterial (e.g., mycobacterial) RNA or DNA, as well as enriched plasmids enriched for a therapeutic nucleic acid. In some embodiments, a “therapeutic nucleic acid-enriched plasmid” refers to a linear or circular plasmid that comprises or is engineered to comprise a greater number of CpG motifs than normally found in mammalian DNA.

Exemplary, non-limiting therapeutic nucleic acid-enriched plasmids are described in, for example, Roman et al. (1997) Nat Med. 3(8):849-54. Modifications of oligonucleotides include, but are not limited to, modifications of the 3′OH or 5′OH group, modifications of the nucleotide base, modifications of the sugar component, and modifications of the phosphate group.

A therapeutic nucleic acid may comprise at least one nucleoside comprising an L-sugar. The L-sugar may be deoxyribose, ribose, pentose, deoxypentose, hexose, deoxyhexose, glucose, galactose, arabinose, xylose, lyxose, or a sugar “analog” cyclopentyl group. The L-sugar may be in pyranosyl or furanosyl form.

Therapeutic nucleic acids generally do not provide for, nor is there any requirement that they provide for, expression of any amino acid sequence encoded by the polynucleotide, and thus the sequence of a therapeutic nucleic acid may be, and generally is, non-coding. Therapeutic nucleic acids may comprise a linear double or single-stranded molecule, a circular molecule, or can comprise both linear and circular segments. Therapeutic nucleic acids may be single-stranded, or may be completely or partially double-stranded.

In some embodiments, a therapeutic nucleic acid for use in a subject method is an oligonucleotide, e.g., consists of a sequence of from about 5 to about 200, from about 10 to about 100, from about 12 to about 50, from about 15 to about 25, from about 5 to about 15, from about 5 to about 10, or from about 5 to about 7 nucleotides in length. In some embodiments, a therapeutic nucleic acid that is less than about 15, less than about 12, less than about 10, or less than about 8 nucleotides in length is associated with a larger molecule, e.g., adsorbed onto an insoluble support, as described below.

In some embodiments, a therapeutic nucleic acid does not provide for expression of a peptide or polypeptide in a eukaryotic cell, e.g., introduction of a therapeutic nucleic acid into a eukaryotic cell does not result in production of a peptide or polypeptide, because the therapeutic nucleic acid does not provide for transcription of an mRNA encoding a peptide or polypeptide. In these embodiments, a therapeutic nucleic acid lacks promoter regions and other control elements necessary for transcription in a eukaryotic cell.

A therapeutic nucleic acid can be isolated from a bacterium, e.g., separated from a bacterial source; synthetic (e.g., produced by standard methods for chemical synthesis of polynucleotides); produced by standard recombinant methods, then isolated from a bacterial source; or a combination of the foregoing. In many embodiments, a therapeutic nucleic acid is purified, e.g., is at least about 80%, at least about 90%, at least about 95%, at least about 98%, at least about 99%, or more, pure. In many embodiments, the purity of the therapeutic nucleic acid is pharmaceutical grade, e.g., greater than 99% pure. In many embodiments, a therapeutic nucleic acid is a synthetic nucleic acid and is purified. In other embodiments, a therapeutic nucleic acid is isolated from a bacterial source; and is purified.

In some embodiments, a therapeutic nucleic acid is part of a larger nucleotide construct (e.g., a plasmid vector, a viral vector, or other such construct). A wide variety of plasmid and viral vector are known in the art, and need not be elaborated upon here. A large number of such vectors has been described in various publications, including, e.g., Current Protocols in Molecular Biology, (F. M. Ausubel, et al., Eds. 1987, and updates). Many vectors are commercially available.

### Therapeutic Nucleic Acids Comprising a CpG Motif

In general, a therapeutic nucleic acid used in a subject method comprise at least one unmethylated CpG motif. The relative position of any CpG sequence in a polynucleotide in certain mammalian species (e.g., rodents) is 5′-CG-3′ (i.e., the C is in the 5′ position with respect to the G in the 3′ position).

In some embodiments, a therapeutic nucleic acid comprises a central palindromic core sequence comprising at least one CpG sequence, where the central palindromic core sequence contains a phosphodiester backbone, and where the central palindromic core sequence is flanked on one or both sides by phosphorothioate backbone-containing polyguanosine sequences.

In other embodiments, a therapeutic nucleic acid comprises one or more TCG sequences at or near the 5′ end of the nucleic acid; and at least two additional CG dinucleotides. In some of these embodiments, the at least two additional CG dinucleotides are spaced three nucleotides, two nucleotides, or one nucleotide apart. In some of these embodiments, the at least two additional CG dinucleotides are contiguous with one another. In some of these embodiments, the therapeutic nucleic acid comprises (TCG)n, where n=one to three, at the 5′ end of the nucleic acid. In other embodiments, the therapeutic nucleic acid comprises (TCG)n, where n=one to three (or more), and where the (TCG)n sequence is flanked by one nucleotide, two nucleotides, three nucleotides, four nucleotides, or five nucleotides, on the 5′ end of the (TCG)n sequence.

In some embodiments, a therapeutic nucleic acid comprises a nucleotide sequence of the formula 5′-Nₘ-(TCG)ₙ-Nₚ-3′, wherein N is any nucleotide, wherein m and p are independently zero or an integer from 1 to about 200, and wherein n is any integer that is 1 or greater. In some embodiments, a therapeutic nucleic acid comprises a nucleotide sequence of the formula 5′-Nₘ-(TCGN_{q})ₙ-Nₚ-3′, wherein N is any nucleotide, wherein m and p are independently zero or an integer from 1 to about 200, wherein q is zero, or an integer from 1 to 5, and wherein n is any integer that is 1 or greater.

Exemplary consensus CpG motifs of therapeutic nucleic acids useful in the invention include, but are not necessarily limited to:
5′-Purine-Purine-(C)-(G)-Pyrimidine-Pyrimidine-3′, in which the therapeutic nucleic acid comprises a CpG motif flanked by at least two purine nucleotides (e.g., GG, GA, AG, AA, II, etc.,) and at least two pyrimidine nucleotides (CC, TT, CT, TC, UU, etc.);
5′-Purine-TCG-Pyrimidine-Pyrimidine-3′;
5′-TCG-N-N-3′; where n is any base;
5′-(TCG)ₙ-3′, where n is any integer that is 1 or greater, e.g., to provide a TCG-based therapeutic nucleic acid (e.g., where n=3, the polynucleotide in some embodiments comprises the sequence 5′-TCGNNTCGNNTCG-3′; SEQ ID NO:1);
5′ Nₘ-(TCG)n-Nₚ-3′, where N is any nucleotide, where m is zero, one, two, or three, where n is any integer that is 1 or greater, and where p is one, two, three, or four;
5′ Nₘ-(TCG)ₙ-Nₚ-3′, where N is any nucleotide, where m is zero to 5, and where n is any integer that is 1 or greater, where p is four or greater, and where the sequence N-N-N-N comprises at least two CG dinucleotides that are either contiguous with each other or are separated by one nucleotide, two nucleotides, or three nucleotides; and
5′-Purine-Purine-CG-Pyrimidine-TCG-3′.

A non-limiting example of a nucleic acid comprising 5′-(TCG)ₙ-3′, where n is any integer that is 1 or greater, is a nucleic acid comprising the sequence 5′ TCGTCGTTTTGTCGTTTTGTCGTT 3′ (SEQ ID NO:2).

Where a nucleic acid comprises a sequence of the formula: 5′-Nₘ-(TCG)ₙ-Nₚ-3′, where N is any nucleotide, where m is zero to 5, and where n is any integer that is 1 or greater, where p is four or greater, and where the sequence N-N-N-N comprises at least two CG dinucleotides that are either contiguous with each other or are separated by one nucleotide, two nucleotides, or three nucleotides, exemplary therapeutic nucleic acids useful in the invention include, but are not necessarily limited to:
(1) a sequence of the formula in which n=2, and Nₚ is NNCGNNCG;
(2) a sequence of the formula in which n=2, and Nₚ is AACGTTCG;
(3) a sequence of the formula in which n=2, and Nₚ is TTCGAACG;
(4) a sequence of the formula in which n=2, and Nₚ is TACGTACG;
(5) a sequence of the formula in which n=2, and Nₚ is ATCGATCG;
(6) a sequence of the formula in which n=2, and Nₚ is CGCGCGCG;
(7) a sequence of the formula in which n=2, and Nₚ is GCCGGCCG;
(8) a sequence of the formula in which n=2, and Nₚ is CCCGGGCG;
(9) a sequence of the formula in which n=2, and Nₚ is GGCGCCCG;
(10) a sequence of the formula in which n=2, and Nₚ is CCCGTTCG;
(11) a sequence of the formula in which n=2, and Nₚ is GGCGTTCG;
(12) a sequence of the formula in which n=2, and Nₚ is TTCGCCCG;
(13) a sequence of the formula in which n=2, and Nₚ is TTCGGGCG;
(14) a sequence of the formula in which n=2, and Nₚ is AACGCCCG;
(15) a sequence of the formula in which n=2, and Nₚ is AACGGGCG;
(16) a sequence of the formula in which n=2, and Nₚ is CCCGAACG; and
(17) a sequence of the formula in which n=2, and Nₚ is GGCGAACG;
   and where, in any of 1-17, m=zero, one, two, or three.

Where a nucleic acid comprises a sequence of the formula: 5′ Nₘ-(TCG)n-Nₚ-3′, where N is any nucleotide, where m is zero, one, two, or three, where n is any integer that is 1 or greater, and where p is one, two, three, or four, exemplary therapeutic nucleic acids useful in the invention include, but are not necessarily limited to:
(1) a sequence of the formula where m=zero, n=1, and Nₚ is T-T-T;
(2) a sequence of the formula where m=zero, n=1, and Nₚ is T-T-T-T;
(3) a sequence of the formula where m=zero, n=1, and Nₚ is C-C-C-C;
(4) a sequence of the formula where m=zero, n=1, and Nₚ is A-A-A-A;
(5) a sequence of the formula where m=zero, n=1, and Nₚ is A-G-A-T;
(6) a sequence of the formula where Nₘ is T, n=1, and Nₚ is T-T-T;
(7) a sequence of the formula where Nₘ is A, n=1, and Nₚ is T-T-T;
(8) a sequence of the formula where Nₘ is C, n=1, and Nₚ is T-T-T;
(9) a sequence of the formula where Nₘ is G, n=1, and Nₚ is T-T-T;
(10) a sequence of the formula where Nₘ is T, n=1, and Nₚ is A-T-T;
(11) a sequence of the formula where Nₘ is A, n=1, and Nₚ is A-T-T; and
(12) a sequence of the formula where Nₘ is C, n=1, and Nₚ is A-T-T.

The core structure of a therapeutic nucleic acid useful in the invention may be flanked upstream and/or downstream by any number or composition of nucleotides or nucleosides. In some embodiments, the core sequence of a therapeutic nucleic acid is at least 6 bases or 8 bases in length, and the complete therapeutic nucleic acid (core sequences plus flanking sequences 5′, 3′ or both) is usually between 6 bases or 8 bases, and up to about 200 bases in length. In some embodiments, a therapeutic nucleic acid ranges in length from about 6 nucleotides to about 200 nucleotides, e.g., from about 6 nucleotides to about 10 nucleotides, from about 10 nucleotides to about 15 nucleotides, from about 15 nucleotides to about 20 nucleotides, from about 10 nucleotides to about 20 nucleotides, from about 20 nucleotides to about 25 nucleotides, from about 25 nucleotides to about 30 nucleotides, from about 30 nucleotides to about 40 nucleotides, from about 40 nucleotides to about 50 nucleotides, from about 50 nucleotides to about 60 nucleotides, from about 60 nucleotides to about 70 nucleotides, from about 70 nucleotides to about 80 nucleotides, from about 80 nucleotides to about 90 nucleotides, from about 90 nucleotides to about 100 nucleotides, from about 100 nucleotides to about 125 nucleotides, from about 125 nucleotides to about 150 nucleotides, from about 150 nucleotides to about 175 nucleotides, or from about 175 nucleotides to about 200 nucleotides in length.

Exemplary DNA-based therapeutic nucleic acids useful in the invention include, but are not necessarily limited to, polynucleotides comprising one or more of the following nucleotide sequences: AGCGCT, AGCGCC, AGCGTT, AGCGTC, AACGCT, AACGCC, AACGTT, AACGTC, GGCGCT, GGCGCC, GGCGTT, GGCGTC, GACGCT, GACGCC, GACGTT, GACGTC, GTCGTC, GTCGCT, GTCGTT, GTCGCC, ATCGTC, ATCGCT, ATCGTT, ATCGCC, TCGTCG, and TCGTCGTCG.

Exemplary DNA-based therapeutic nucleic acids useful in the invention include, but are not necessarily limited to, polynucleotides comprising the following octameric nucleotide sequences: AGCGCTCG, AGCGCCCG, AGCGTTCG, AGCGTCCG, AACGCTCG, AACGCCCG, AACGTTCG, AACGTCCG, GGCGCTCG, GGCGCCCG, GGCGTTCG, GGCGTCCG, GACGCTCG, GACGCCCG, GACGTTCG, and GACGTCCG.

A therapeutic nucleic acid useful in carrying out a subject method can comprise one or more of any of the above CpG motifs. For example, a therapeutic nucleic acid useful in the invention can comprise a single instance or multiple instances (e.g., 2, 3, 5 or more) of the same CpG motif. Alternatively, a therapeutic nucleic acid can comprise multiple CpG motifs (e.g., 2, 3, 5 or more) where at least two of the multiple CpG motifs have different consensus sequences, or where all CpG motifs in the therapeutic nucleic acid have different consensus sequences.

A therapeutic nucleic acid useful in the invention may or may not include palindromic regions. If present, a palindrome may extend only to a CpG motif, if present, in the core hexamer or octamer sequence, or may encompass more of the hexamer or octamer sequence as well as flanking nucleotide sequences.

In some embodiments, a combination of two or more therapeutic nucleic acids, each having different nucleotide sequences, will be administered. In some embodiments, a mixture of two or more therapeutic nucleic acid comprises a first therapeutic nucleic acid comprising a first nucleotide sequence; and a second therapeutic nucleic acid comprising a second nucleotide sequence, where the second nucleotide sequence differs from the first nucleotide sequence by from one to about 10 bases, or by from about one to about 20 bases. In some embodiments, a therapeutic nucleic acid mixture comprises more than two different therapeutic nucleic acids (e.g., three, four, five, or more different therapeutic nucleic acids), each of which differs in nucleotide sequence from the other therapeutic nucleic acids in the mixture by from about one to about 10 bases, or from about one to about 20 bases. Thus, in some embodiments, a subject method for treating IBS comprises administering a mixture of two or more therapeutic nucleic acids, each having a different nucleotide sequence.

### MODIFICATIONS

A therapeutic nucleic acid suitable for use in a subject method can be modified in a variety of ways. For example, a therapeutic nucleic acid can comprise backbone phosphate group modifications (e.g., methylphosphonate, phosphorothioate, phosphoroamidate and phosphorodithioate internucleotide linkages), which modifications can, for example, enhance their stability in vivo, making them particularly useful in therapeutic applications. A particularly useful phosphate group modification is the conversion to the phosphorothioate or phosphorodithioate forms of a therapeutic nucleic acid. Phosphorothioates and phosphorodithioates are more resistant to degradation in vivo than their unmodified oligonucleotide counterparts, increasing the half-lives of the therapeutic nucleic acids and making them more available to the subject being treated.

Other modified therapeutic nucleic acids encompassed by the present invention include therapeutic nucleic acids having modifications at the 5′ end, the 3′ end, or both the 5′ and 3′ ends. For example, the 5′ and/or 3′ end can be covalently or non-covalently associated with a molecule (either nucleic acid, non-nucleic acid, or both) to, for example, increase the bio-availability of the therapeutic nucleic acid, increase the efficiency of uptake where desirable, facilitate delivery to cells of interest, and the like. Exemplary molecules for conjugation to a therapeutic nucleic acid include, but are not necessarily limited to, cholesterol, phospholipids, fatty acids, sterols, oligosaccharides, polypeptides (e.g., immunoglobulins), peptides, antigens (e.g., peptides, small molecules, etc.), linear or circular nucleic acid molecules (e.g., a plasmid), insoluble supports, and the like.

A therapeutic nucleic acid may be associated with (complexed with or encapsulated by) a microcarrier. See, e.g. U.S. Patent Publication No. 20030133988. For example, a therapeutic nucleic acid 3-10 nucleotides, 3-8 nucleotides, or 3-6 nucleotides in length is linked to an insoluble microcarrier (MC) which may be either biodegradable or nonbiodegradable. The therapeutic nucleic acid may be covalently or non-covalently linked to the microcarrier in the complex, and the therapeutic nucleic acid may be modified to facilitate complex formation. Microcarriers are typically solid phase microcarriers, although liquid phase microcarriers (e.g., an oil in water emulsion comprising a polymer or oil, preferably a biodegradable polymer or oil) can also be used. Microcarriers are generally less than about 150, 120 or 100 μm in size, less than about 50-60 μm in size, about 10 nm to about 10 μm in size, or about 25 nm to 5 μm in size.

A therapeutic nucleic acid is in some embodiments linked (e.g., conjugated, covalently linked, non-covalently associated with, or adsorbed onto) an insoluble support. An exemplary, non-limiting example of an insoluble support is cationic poly(D,L-lactide-co-glycolide).

Additional therapeutic nucleic acid conjugates, and methods for making same, are known in the art and described in, for example, WO 98/16427 and WO 98/55495. Thus, the term “therapeutic nucleic acid” includes conjugates comprising a therapeutic nucleic acid.

A polypeptide, e.g., a therapeutic polypeptide, may be conjugated directly or indirectly, e.g., via a linker molecule, to a therapeutic nucleic acid. A wide variety of linker molecules are known in the art and can be used in the conjugates. The linkage from the peptide to the oligonucleotide may be through a peptide reactive side chain, or the N- or C-terminus of the peptide. Linkage from the oligonucleotide to the peptide may be at either the 3′ or 5′ terminus, or internal. A linker may be an organic, inorganic, or semi-organic molecule, and may be a polymer of an organic molecule, an inorganic molecule, or a co-polymer comprising both inorganic and organic molecules.

If present, the linker molecules are generally of sufficient length to permit oligonucleotides and/or polynucleotides and a linked polypeptide to allow some flexible movement between the oligonucleotide and the polypeptide. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to oligonucleotides may be used in light of this disclosure.

Peptides may be synthesized chemically or enzymatically, may be produced recombinantly, may be isolated from a natural source, or a combination of the foregoing. Peptides may be isolated from natural sources using standard methods of protein purification known in the art, including, but not limited to, a liquid chromatography method (e.g., HPLC), size exclusion chromatography, gel electrophoresis (one-dimensional or two-dimensional), affinity chromatography, or other purification technique. One may employ solid phase peptide synthesis techniques, where such techniques are known to those of skill in the art. See Jones, The Chemical Synthesis of Peptides (Clarendon Press, Oxford) (1994). Generally, in such methods a peptide is produced through the sequential additional of activated monomeric units to a solid phase bound growing peptide chain. Well-established recombinant DNA techniques can be employed for production of peptides.

### Formulations, Dosages, and Routes of Administration

In a subject method, an active agent, i.e., a therapeutic nucleic acid, is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, the active agents may be administered to the host using any convenient means capable of resulting in the desired therapeutic effect. Thus, the agents can be incorporated into a variety of formulations for therapeutic administration. More particularly, the active agents can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In the methods of the subject invention, an effective amount of the active agent(s) is administered to the subject, where “effective amount” means a dosage sufficient to produce the desired result, e.g., an improvement in a disease condition or the symptoms associated therewith of the IBS condition being treated, e.g., bloating, pain, etc. The active agent may be administered to the host using any convenient means capable of producing the desired result. Thus, the active agent can be incorporated into a variety of formulations for therapeutic administration. More particularly, the active agent of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the active agent can be achieved in various ways, including oral, buccal, rectal, parenteral, intraperitoneal, intradermal, transdermal, intracheal, etc., administration. In pharmaceutical dosage forms, the active agent may be administered alone or in combination with other pharmaceutically active compounds.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for the unit dosage forms depend on the particular compound employed (e.g., the particular therapeutic nucleic acid) and the effect to be achieved, and the pharmacodynamics associated with each compound in the host.

The dose of therapeutic nucleic acid administrated to a subject, in the context of the present invention, should be sufficient to effect a beneficial therapeutic response in the subject over time, or to alleviate symptoms. Thus, a therapeutic nucleic acid is administered to a patient in an amount sufficient to alleviate, reduce, cure or at least partially arrest symptoms of IBS. An amount adequate to accomplish this is defined as a “therapeutically effective dose.”

Although the dosage used will vary depending on the clinical goals to be achieved, a suitable dosage range is one which provides up to about 1 μg, to about 1,000 μg, to about 5,000 μg, to about 10,000 μg, to about 25,000 μg, to about 50,000 μg, to about 100,000 μg, or to about 500,000 μg of therapeutic nucleic acid per ml of carrier (or per gram of carrier, or per other unit dosage form) in a single dosage. A unit dosage form of a therapeutic nucleic acid contains from about 1 μg to about 500 mg (e.g., from about 1 μg to about 5 μg, from about 5 μg to about 10 μg, from about 10 μg to about 50 μg, from about 50 μg to about 100 μg, from about 100 μg to about 250 μg, from about 250 μg to about 500 μg, from about 500 μg to about 1 mg, from about 1 mg to about 10 mg, from about 10 mg to about 50 mg, from about 50 mg to about 100 mg, from about 100 mg to about 250 mg, from about 250 mg to about 500 mg of a therapeutic nucleic acid. For example, a unit dosage form of a food product (e.g., a lozenge, a piece of chewing gum, a food bar, a serving size of a food product, etc.), or a tablet, a capsule, etc., comprises from about 1 μg to about 500 mg of a therapeutic nucleic acid.

In some embodiments, a therapeutic nucleic acid is delivered by inhalation. A therapeutic nucleic acid composition may be administered to an individual by means of a pharmaceutical delivery system for the inhalation route (oral, intratracheal, intranasal). Thus, therapeutic nucleic acid composition may be formulated in a form suitable for administration by inhalation. The pharmaceutical delivery system is one that is suitable for respiratory therapy by topical administration of a therapeutic nucleic acid to mucosal linings of the bronchi. This invention can utilize a system that depends on the power of a compressed gas to expel the agent from a container. An aerosol or pressurized package can be employed for this purpose.

As used herein, the term “aerosol” is used in its conventional sense as referring to very fine liquid or solid particles carries by a propellant gas under pressure to a site of therapeutic application. When a pharmaceutical aerosol is employed in this invention, the aerosol contains the agent, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Examples of suitable propellants include, but is not limited to, hydrocarbons or other suitable gas. In the case of the pressurized aerosol, the dosage unit may be determined by providing a value to deliver a metered amount.

A therapeutic nucleic acid composition can also be delivered to the respiratory tract with a nebulizer, which is an instrument that generates very fine liquid particles of substantially uniform size in a gas. In many embodiments, a liquid containing a therapeutic nucleic acid is dispersed as droplets. The small droplets can be carried by a current of air through an outlet tube of the nebulizer. The resulting mist penetrates into the respiratory tract of the patient.

A powder composition containing a therapeutic nucleic acid, with or without a lubricant, carrier, or propellant, can be administered to a mammal. This embodiment of the invention can be carried out with a conventional device for administering a powder pharmaceutical composition by inhalation. For example, a powder mixture of a therapeutic nucleic acid and a suitable powder base such as lactose or starch may be presented in unit dosage form in for example capsular or cartridges, e.g. gelatin, or blister packs, from which the powder may be administered with the aid of an inhaler.

There are several different types of inhalation methodologies which can be employed in connection with the present invention. A therapeutic nucleic acid can be formulated in basically three different types of formulations for inhalation. First, a therapeutic nucleic acid can be formulated with low boiling point propellants. Such formulations are generally administered by conventional meter dose inhalers (MDI's). However, conventional MDI's can be modified so as to increase the ability to obtain repeatable dosing by utilizing technology which measures the inspiratory volume and flow rate of the patient as discussed within U.S. Pat. Nos. 5,404,871 and 5,542,410.

Alternatively, a therapeutic nucleic acid can be formulated in aqueous or ethanolic solutions and delivered by conventional nebulizers. In some embodiments, such solution formulations are aerosolized using devices and systems such as disclosed within U.S. Pat. Nos. 5,497,763; 5,544,646; 5,718,222; and 5,660,166.

Furthermore, a therapeutic nucleic acid can be formulated into dry powder formulations. Such formulations can be administered by simply inhaling the dry powder formulation after creating an aerosol mist of the powder. Technology for carrying such out is described within U.S. Pat. No. 5,775,320 and U.S. Pat. No. 5,740,794.

Formulations suitable for intranasal administration include nasal sprays, nasal drops, aerosol formulations; and the like.

### Combination Therapies

In one embodiment, the present invention provides a method of treating IBS in an individual, involving administering effective amounts of a therapeutic nucleic acid and at least a second therapeutic agent, the method comprising to the individual: a) a therapeutic nucleic acid in an amount of from about 1 μg to about 500 mg; and b) a second therapeutic agent selected from an anti-diarrheal agent, a serotonin 5HT4 agonist, a serotonin 5HT3 antagonist, a laxative, an anti-depressant, and an anti-spasmodic agent, for the desired treatement duration, to reduce at least one symptom of IBS in the individual.

In particular embodiments, e.g., where an individual having IBS experiences diarrhea, a subject method comprises administering a dosage of a therapeutic nucleic acid containing an amount of from about 1 μg to about 500 mg therapeutic nucleic acid; and a dosage of dicyclomine HCl containing an amount from about 10 mg to about 40 mg orally qid, for the desired treatment duration. In particular embodiments, the therapeutic nucleic acid

In particular embodiments, e.g., where an individual having IBS experiences diarrhea, a subject method comprises administering a dosage of a therapeutic nucleic acid containing an amount of from about 1 μg to about 500 mg therapeutic nucleic acid; and a dosage of hyoscyamine sulfate containing an amount from about 0.125 to about 0.25 mg orally every 4 hours, for the desired treatment duration.

In particular embodiments, e.g., where an individual having IBS experiences diarrhea, a subject method comprises administering a dosage of a therapeutic nucleic acid containing an amount of from about 1 μg to about 500 mg therapeutic nucleic acid; and a dosage of Lomotil (diphenoxylate HCl+atropine sulfate) orally qid, for the desired treatment duration.

In particular embodiments, e.g., where an individual having IBS experiences diarrhea, a subject method comprises administering a dosage of a therapeutic nucleic acid containing an amount of from about 1 μg to about 500 mg therapeutic nucleic acid; and a dosage of loperamide containing an amount of 4 mg orally after first loose stool, the 2 mg after each subsequent stool, for the desired treatment duration.

In particular embodiments, e.g., where an individual having IBS experiences constipation, a subject method comprises administering a dosage of a therapeutic nucleic acid containing an amount of from about 1 μg to about 500 mg therapeutic nucleic acid; and a dosage of Tegaserod containing an amount of 6 mg orally bid, for the desired treatment duration.

In some embodiments, any of the above-described methods is modified such that the therapeutic nucleic acid comprises the nucleotide sequence 5′-(TCG)ₙ-3′, where n=1 or greater, e.g., n=1-3. In some embodiments, any of the above-described methods is modified such that the therapeutic nucleic acid comprises two or more 5′-CG-3′ motifs. In some embodiments, any of the above-described methods is modified such that the therapeutic nucleic acid comprises the nucleotide sequence 5′ Nₘ-(TCG)n-Nₚ-3′, where N is any nucleotide, where m is zero, one, two, or three, where n is any integer that is 1 or greater (e.g., n=1-3), and where p is one, two, three, or four. In some embodiments, any of the above-described methods is modified such that the therapeutic nucleic acid is formulated with one or more food-grade carriers, e.g., is provided as a food product. In some embodiments, any of the above-described methods is modified such that the therapeutic nucleic acid is formulated as a nutraceutical.

Nutraceutical Formulations; Food Products

In some embodiments, the therapeutic nucleic acid is formulated with one or more food-grade components, e.g., a dosage form is a nutraceutical or a food product. The term “nutraceutical formulation” refers to a food or part of a food that offers medical and/or health benefits including prevention or treatment of disease. Nutraceutical products range from isolated nutrients, dietary supplements and diets, to genetically engineered designer foods, functional foods, herbal products and processed foods such as cereal, soup and beverages. The term “functional foods,” refers to foods that include “any modified food or food ingredients that may provide a health benefit beyond the traditional nutrients it contains.” Thus, by definition, pharmaceutical compositions comprising a therapeutic nucleic acid include nutraceuticals. Also by definition, pharmaceutical compositions comprising a therapeutic nucleic acid include compositions comprising a therapeutic nucleic acid and a food-grade component. A therapeutic nucleic acid may be added to food products to provide a health benefit.

Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; lozenges; candies; and the like. These foods are enhanced by the inclusion of a therapeutic nucleic acid. For example, in the treatment of an IBS, the normal diet of a patient may be supplemented by a therapeutic nucleic acid nutraceutical formulation taken on a regular basis, e.g., at meal times, before meals, or after meals.

The present invention provides compositions (e.g., nutraceutical compositions) comprising a therapeutic nucleic acid and a food-grade pharmaceutically acceptable excipient. In many embodiments, therapeutic nucleic acid nutraceutical compositions include one or more components found in food products. Thus, the instant invention provides a food composition and products comprising a therapeutic nucleic acid and a food component. Suitable components include, but are not limited to, mono- and disaccharides; carbohydrates; proteins; amino acids; fatty acids; lipids; stabilizers; preservatives; flavoring agents; coloring agents; sweeteners; antioxidants, chelators, and carriers; texturants; nutrients; pH adjusters; emulsifiers; stabilizers; milk base solids; edible fibers; and the like. The food component can be isolated from a natural source, or can be synthesized. All components are food-grade components fit for human consumption.

Examples of suitable monosaccharides include sorbitol, mannitol, erythrose, threose, ribose, arabinose, xylose, ribulose, glucose, galactose, mannose, fructose, and sorbose. Non-limiting examples of suitable disaccharides include sucrose, maltose, lactitol, maltitol, maltulose, and lactose.

Suitable carbohydrates include oligosaccharides, polysaccharides, and/or carbohydrate derivatives. As used herein, the term “oligosaccharide” refers to a digestible linear molecule having from 3 to 9 monosaccharide units, wherein the units are covalently connected via glycosidic bonds. As used herein, the term “polysaccharide” refers to a digestible (i.e., capable of metabolism by the human body) macromolecule having greater than 9 monosaccharide units, wherein the units are covalently connected via glycosidic bonds. The polysaccharides may be linear chains or branched. Carbohydrate derivatives, such as a polyhydric alcohol (e.g., glycerol), may also be utilized as a complex carbohydrate herein. As used herein, the term “digestible” in the context of carbohydrates refers to carbohydrate that are capable of metabolism by enzymes produced by the human body. Examples of polysaccharides non-digestible carbohydrates are resistant starches (e.g., raw corn starches) and retrograded amyloses (e.g., high amylose corn starches). Non-limiting examples carbohydrates include raffinoses, stachyoses, maltotrioses, maltotetraoses, glycogens, amyloses, amylopectins, polydextroses, and maltodextrins.

Suitable fats include, but are not limited to, triglycerides, including short-chain (C₂-C₄) and long-chain triglycerides (C₁₆-C₂₂).

Suitable texturants (also referred to as soluble fibers) include, but are not limited to, pectin (high ester, low ester); carrageenan; alginate (e.g., alginic acid, sodium alginate, potassium alginate, calcium alginate); guar gum; locust bean gum; psyllium; xanthan gum; gum arabic; fructo-oligosaccharides; inulin; agar; and functional blends of two or more of the foregoing.

Suitable emulsifiers include, but are not limited to, propylene glycol monostearate (PGMS), sodium stearoyl lactylate (SSL), calcium stearoyl lactylate (CSL), monoglycerides, diglycerides, monodiglycerides, polyglycerol esters, lactic acid esters, polysorbate, sucrose esters, diacetyl tartaric acid esters of mono-diglycerides (DATEM), citric acid esters of monoglycerides (CITREM) and combinations thereof. Additional suitable emulsifiers include DIMODAN distilled monoglycerides, including DIMODAN™ B 727 and DIMODAN™ PV, GRINDSTED™ CITREM, GRINDSTED™ GA, GRINDSTED™ PS such as GRINDSTED™ PS 100, GRINDSTED™ PS 200, GRINDSTED™ PS 300, GRINDSTED™ PS 400; RYLO™ (manufactured and distributed by DANISCO CULTOR), including RYLO™ AC, RYLO™ CI, RYLO™ LA, RYLO™ MD, RYLO™ MG, RYLO™ PG, RYLO™ PR, RYLO™ SL, RYLO™ SO, RYLO™ TG; and combinations thereof.

Edible fibers include polysaccharides, oligosaccharides, lignin and associated plant substances. Suitable edible fibers include, but are not limited to, sugar beet fiber, apple fiber, pea fiber, wheat fiber, oat fiber, barley fiber, rye fiber, rice fiber, potato fiber, tomato fiber, other plant non-starch polysaccharide fiber, and combinations thereof.

Suitable flavoring agents include natural and synthetic flavors, “brown flavorings” (e.g., coffee, tea); dairy flavorings; fruit flavors; vanilla flavoring; essences; extracts; oleoresins; juice and drink concentrates; flavor building blocks (e.g., delta lactones, ketones); and the like; and combinations of such flavors. Examples of botanic flavors include, for example, tea (e.g., preferably black and green tea), aloe vera, guarana, ginseng, ginkgo, hawthorn, hibiscus, rose hips, chamomile, peppermint, fennel, ginger, licorice, lotus seed, schizandra, saw palmetto, sarsaparilla, safflower, St. John's Wort, curcuma, cardamom, nutmeg, cassia bark, buchu, cinnamon, jasmine, haw, chrysanthemum, water chestnut, sugar cane, lychee, bamboo shoots, vanilla, coffee, and the like.

Suitable sweeteners include, but are not limited to, alitame; dextrose; fructose; lactilol; polydextrose; xylitol; xylose; aspartame, saccharine, cyclamates, acesulfame K, L-aspartyl-L-phenylalanine lower alkyl ester sweeteners, L-aspartyl-D-alanine amides; L-aspartyl-D-serine amides; L-aspartyl-hydroxymethyl alkane amide sweeteners; L-aspartyl-1-hydroxyethylalkane amide sweeteners; and the like.

Suitable anti-oxidants include, but are not limited to, tocopherols (natural, synthetic); ascorbyl palmitate; gallates; butylated hydroxyanisole (BHA); butylated hydroxytoluene (BHT); tert-butyl hydroquinone (TBHQ); and the like.

Suitable nutrients include vitamins and minerals, including, but not limited to, niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iron, zinc, copper, calcium, phosphorous, iodine, chromium, molybdenum, and fluoride.

Suitable coloring agents include, but are not limited to, FD&C dyes (e.g., yellow #5, blue #2, red #40), FD&C lakes; Riboflavin; β-carotene; natural coloring agents, including, for example, fruit, vegetable, and/or plant extracts such as grape, black currant, aronia, carrot, beetroot, red cabbage, and hibiscus.

Exemplary preservatives include sorbate, benzoate, and polyphosphate preservatives.

Suitable emulsifiers include, but are not limited to, diglycerides; monoglycerides; acetic acid esters of mono- and diglycerides; diacetyl tartaric acid esters of mono- and diglycerides; citric acid esters of mono- and diglycerides; lactic acid esters of mono- and diglycerides; fatty acids; polyglycerol esters of fatty acids; propylene glycol esters of fatty acids; sorbitan monostearates; sorbitan tristearates; sodium stearoyl lactylates; calcium stearoyl lactylates; and the like.

Suitable agents for pH adjustment include organic as well as inorganic edible acids. The acids can be present in their undissociated form or, alternatively, as their respective salts, for example, potassium or sodium hydrogen phosphate, potassium or sodium dihydrogen phosphate salts. Exemplary acids are edible organic acids which include citric acid, malic acid, fumaric acid, adipic acid, phosphoric acid, gluconic acid, tartaric acid, ascorbic acid, acetic acid, phosphoric acid and mixtures thereof.

Therapeutic nucleic acids are present in the food product/nutraceutical formulation in an amount of from about 0.01% to about 30% by weight, e.g., from about 0.01% to about 0.1%, from about 0.1% to about 0.5%, from about 0.5% to about 1.0%, from about 1.0% to about 2.0%, from about 2.0% to about 5%, from about 5% to about 7%, from about 7% to about 10%, from about 10% to about 15%, from about 15% to about 20%, from about 20% to about 25%, or from about 25% to about 30% by weight. In some embodiments, the therapeutic nucleic acid present in the food product is homogenous, e.g., substantially all the therapeutic nucleic acids in the food product have the same sequence. In other embodiments, the therapeutic nucleic acids in the food product comprise therapeutic nucleic acids of two or more different nucleotide sequences.

Where the food product is a beverage, the food product generally contains, by volume, more than about 50% water, e.g., from about 50% to about 60%, from about 60% to about 95% water, e.g., from about 60% to about 70%, from about 70% to about 80%, from about 80% to about 90%, or from about 90% to about 95% water.

Where the food product is a solid or semi-solid food product, e.g., a bar, tablet, solid candy, lozenge, etc., the food product generally contains, by volume, less than about 15% water, e.g., from about 2% to about 5%, from about 5% to about 7%, from about 7% to about 10%, from about 10% to about 12%, or from about 12% to about 15% water.

In some embodiments, the food product is essentially dry, e.g., comprises less than about 5%, water.

Monosaccharides, disaccharides, and complex carbohydrates, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each. Soluble fibers, edible fibers, and emulsifiers, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each.

Other components discussed above, if present, are present in amounts ranging from about 0.001% to about 5% by weight of the composition.

### Kits

Kits with unit doses of the active agent(s) (e.g., therapeutic nucleic acid), usually in oral or injectable doses and often in a storage stable formulation, are provided. Preferred active agents and unit doses are those described herein above. In such kits, in addition to the containers containing the unit doses will be an informational package insert describing the use agent(s) in treating IBS. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, compact disc (CD), etc., on which the information has been recorded. Other suitable media include, audiovisual media, e.g., digital versatile disk (DVD), videotape, and the like. Yet another means that may be present is a website address which may be used via the Internet to access the information at a removed site. Any convenient means may be present in the kits.

### Subjects Suitable for Treatment

The subject methods are suitable for treating any individual who has been diagnosed as having IBS. Children having IBS, as well as adults having IBS, are suitable for treatment with a subject method. Pregnant women having IBS are suitable for treatment, as are women in general who have IBS. Any individual who has been diagnosed as having IBS, and who has failed to respond to treatment with a therapeutic agent other than a therapeutic nucleic acid is also suitable for treatment with a subject method. Any individual who has been diagnosed as having IBS, who has responded to treatment with a therapeutic agent other than a therapeutic nucleic acid, and who has relapsed (e.g., experienced a recurrence of IBS) is also suitable for treatment with a subject method. Individuals who have been diagnosed as having IBS, and who are currently asymptomatic are suitable for treatment with a subject method, to reduce the risk of recurrence of symptoms of IBS.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating irritable bowel syndrome (IBS) in an individual, the method comprising administering to the individual an effective amount of a therapeutic nucleic acid to reduce at least one symptom of IBS in the individual.

2. The method of claim 1, wherein the therapeutic nucleic acid comprises a nucleotide sequence of the formula 5′-CG-3′.

3. The method of claim 2, wherein the therapeutic nucleic acid comprises a nucleotide sequence of the formula 5′ (TCG)ₙ-3′, where n is any integer that is 1 or greater.

4. The method of claim 2, wherein the therapeutic nucleic acid comprises a nucleotide sequence of the formula 5′-Nₘ-(TCG)ₙ-Nₚ-3′, wherein N is any nucleotide, wherein m and p are independently zero or 1-200, and wherein n is any integer that is 1 or greater.

5. The method of claim 1, wherein the therapeutic nucleic acid is administered via a gastroenteral route.

6. The method of claim 5, wherein the gastroenteral route is oral, intranasal, intragastric or rectal.

7. The method of claim 1, wherein the therapeutic nucleic acid is administered by a systemic route.

8. The method of claim 7, wherein the systemic route is intradermal, intramuscular, subcutaneous or intravenous.

9. The method of claim 1, further comprising administering a serotonin 5HT3 antagonist.

10. The method of claim 1, further comprising administering a laxative.

11. The method of claim 1, further comprising administering an antispasmodic agent.

12. The method of claim 1, further comprising administering an antidepressant.

13. The method of claim 1, further comprising administering an antidiarrheal agent.

14. The method of claim 1, wherein the therapeutic nucleic acid is formulated with at least one food-grade carrier.

15. The method of claim 14, wherein the food-grade carrier is selected from the group consisting of olive oil, an emulsifier, a soluble fiber, a flavoring agent, a coloring agent, an edible fiber, and a sweetener.

16. The method of claim 15, wherein the soluble fiber is selected from the group consisting of pectin, carrageenan, alginate, guar gum, locust bean gum, psyllium, xanthan gum, gum arabic, fructo-oligosaccharides, inulin, and agar.

17. The method of claim 15, wherein the emulsifier is selected from the group consisting of propylene glycol monostearate, sodium stearoyl lactylate, calcium stearoyl lactylate, a monoglyceride, a diglyceride, a mono-diglyceride a polyglycerol ester, a lactic acid ester, polysorbate, a sucrose ester, a diacetyl tartaric acid ester of mono-diglycerides, and a citric acid ester of monoglycerides.
